(19) United States
(12) Patent Application Publication
(10) Pub. No.: US 2026/0345960 A1
(43) Pub. Date: Nov. 5, 2026

(54) ADJUSTABLE STIFFNESS GUIDEWIRES

(71) Applicant: Boston Scientific Scimed, Inc., Maple Grove, MN (US)

(72) Inventors: Jessica Rincon Nieto, Marlborough, MA (US); Robb Morse Gavalis, Westborough, MA (US)

(73) Assignee: Boston Scientific Scimed, Inc., Maple Grove, MN (US)

(21) Appl. No.: 19/664,592

(22) Filed: Apr. 30, 2026

Related U.S. Application Data
(60) Provisional application No. 63/800,245, filed on May 5, 2025.

(51) Int. Cl. A61M 25/09

(52) U.S. Cl. CPC A61M 25/09; A61M 2025/09083; A61M 2025/0915

(57) ABSTRACT

A guidewire has an outer member and inner member, where the outer member's distal end is fixed to the inner member's distal end to prevent relative translation. An adjustment mechanism selectively applies compressive force to the outer member's proximal end, increasing guidewire stiffness. The outer member can be a coiled member or distinct annular rings. The adjustment mechanism options include reverse-action pliers with U-shaped jaw cutouts and handles that spread the jaws when squeezed together, or a threaded fastener engaging with a threaded section of the inner member. The distal connection between members can be achieved through crimping or welding. The adjustment mechanism can be removable to allow device exchanges over the guidewire. When compression is applied to the outer member's proximal end, it creates tension in the inner member's proximal end.

### CROSS-REFERENCE TO RELATED APPLICATIONS

This application claims the benefit of U.S. Provisional Patent Application Ser. No. 63/800,245 filed on May 5, 2025, the disclosure of which is incorporated herein by reference.

### TECHNICAL FIELD

The present disclosure pertains to medical devices, and methods for manufacturing medical devices. More particularly, the present disclosure pertains to elongated intracorporeal medical devices such as guidewires, and methods for manufacturing and using such devices.

### BACKGROUND

A wide variety of intracorporeal medical devices have been developed for medical use including intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, method, and use alternatives for medical devices, including but not limited to medical devices such as guidewires. An example may be found in a guidewire. The guidewire includes an outer member and an inner member that extends within the outer member. A distal end of the outer member is constrained from translating relative to a distal end of the inner member. An adjustment mechanism is adapted to selectively apply a compressive force to a proximal end of the outer member. The guidewire increases in stiffness when the adjustment mechanism applies a compressive force to the proximal end of the outer member.

Alternatively or additionally to any example above, applying a compressive force to the proximal end of the outer member may also place a tensile force on the proximal end of the inner member.

Alternatively or additionally to any example above, the distal end of the outer member may be crimped onto the distal end of the inner member.

Alternatively or additionally to any example above, the distal end of the outer member may also be constrained from rotating relative to the distal end of the inner member.

Alternatively or additionally to any example above, the distal end of the outer member may be welded to the distal end of the inner member.

Alternatively or additionally to any example above, the outer member may include an outer coiled member.

Alternatively or additionally to any example above, the outer member may include a plurality of distinct annular rings disposed about the inner member.

Alternatively or additionally to any example above, the adjustment mechanism may include a removable adjustment mechanism.

Alternatively or additionally to any example above, the adjustment mechanism may include reverse-action pliers.

Alternatively or additionally to any example above, may include first and second jaws each including a U-shaped cutout to accommodate the inner member therethrough.

Alternatively or additionally to any example above, the reverse-action pliers may further include first and second handles, where moving the first and second handles closer together causes the first and second jaws to move farther apart.

Alternatively or additionally to any example above, the inner member may include a threaded section and the adjustment mechanism may include a threaded fastener that is threadedly engaged with the threaded section, such that rotation of the adjustment mechanism adjusts compression of the outer member.

Alternatively or additionally to any example above, the threaded fastener may be textured to improve grip.

Another example may be found in a guidewire. The guidewire includes an outer coiled member and an inner member that extends within the outer coiled member. A distal end of the outer coiled member is constrained from translating relative to a distal end of the inner member. An adjustment mechanism is adapted to urge a proximal end of the outer coiled member distally relative to the proximal end of the inner member in order to increase a stiffness of the guidewire and to allow the proximal end of the outer coiled member to move proximally relative to the proximal end of the inner member in order to decrease the stiffness of the guidewire.

Alternatively or additionally to any example above, the distal end of the outer coiled member may be constrained from rotating relative to the distal end of the inner member.

Alternatively or additionally to any example above, the adjustment mechanism may include reverse-action pliers including first and second jaws each including a U-shaped cutout to accommodate the inner member therethrough and first and second handles. Moving the first and second handles closer together causes the first and second jaws to move farther apart.

Alternatively or additionally to any example above, the inner member may include a threaded section and the adjustment mechanism may include a threaded fastener that is threadedly engaged with the threaded section, such that rotation of the adjustment mechanism adjusts compression of the outer coiled member.

Another example may be found in a guidewire. The guidewire includes an outer coiled member and an inner member that extends within the outer coiled member. A distal end of the outer coiled member is welded to a distal end of the inner member. A removable adjustment mechanism is adapted to selectively apply a compressive force to a proximal end of the outer coiled member and to be removed from the guidewire to accommodate device exchanges over the guidewire. The guidewire increases in stiffness when the removable adjustment mechanism applies a compressive force to the proximal end of the outer member.

Alternatively or additionally to any example above, the removable adjustment mechanism may include a first pliers member including a first jaw with a U-shaped cutout and a first handle and a second pliers member including a second jaw with a second U-shaped cutout and a second handle. The second pliers member may be pivotably secured relative to the first pliers member. Urging the first and second handles towards each other may cause the first and second jaws to move away from each other.

Alternatively or additionally to any example above, the removable adjustment mechanism may include a first member that is configured to engage a proximal end of the inner member and that includes one or more finger loops, and a second member that is configured to engage a proximal end of the outer member and that includes one or more finger loops. Moving the first member and the second member closer together applies a compressive force to the outer member and a tensile force to the inner member.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense.

### BRIEF DESCRIPTION OF THE DRAWINGS

The drawings illustrate the design and utility of configurations of the present disclosure. It should be noted that the figures are not drawn to scale and that elements of similar structures or functions are represented by like reference numerals throughout the figures. The drawings are not to be considered as limiting the scope of the disclosure. The present disclosure is described and explained with additional specificity and detail through the use of the accompanying drawings, in which:

FIG. 1 is a schematic view showing an illustrative guidewire;

FIG. 2 is an enlarged view of a portion of the illustrative guidewire of FIG. 1;

FIG. 3 is a schematic view showing an illustrative guidewire;

FIG. 4 is an enlarged view of a portion of the illustrative guidewire of FIG. 3;

FIG. 5 is a schematic view of an illustrative guidewire including an adjustment mechanism;

FIG. 6 is a schematic view of an illustrative guidewire including an outer member in a relaxed configuration;

FIG. 7 is a schematic view of the illustrative guidewire of FIG. 6, shown with the outer member in a compressed configuration;

FIG. 8 is an enlarged view of an illustrative guidewire in which outer member is not constrained relative to inner member;

FIG. 9 is an enlarged view of an illustrative guidewire in which outer member is constrained relative to inner member;

FIG. 10 is a schematic view of an illustrative removable adjustment mechanism engaged with an illustrative guidewire;

FIG. 11 is an enlarged view of a portion of the illustrative removable adjustment mechanism engaged with the illustrative guidewire of FIG. 10;

FIG. 12 is a schematic view of an illustrative guidewire in which the outer member includes a plurality of annular rings;

FIG. 13 is a schematic view of one of the annular rings shown in FIG. 12; and

FIG. 14 is a schematic view of an illustrative removable adjustment mechanism engaged with an illustrative guidewire.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar elements in different drawings are numbered the same. The drawings, which are not necessarily to scale, depict illustrative configurations and are not intended to limit the scope of the disclosure.

In some instances, a guidewire includes an outer member and an inner member that extends within the outer member. A distal end of the outer member is constrained from translating relative to a distal end of the inner member. However, a proximal end of the outer member is not constrained from translating relative to a proximal end of the inner member. An adjustment mechanism is adapted to selectively apply a compressive force to the proximal end of the outer member. The guidewire increases in stiffness when the adjustment mechanism applies a compressive force to the proximal end of the outer member.

In some cases, applying a compressive force to the proximal end of the outer member may also place a tensile force on the proximal end of the inner member. In some cases, the distal end of the outer member may be crimped onto the distal end of the inner member. In some cases, the distal end of the outer member may also be constrained from rotating relative to the distal end of the inner member. As an example, the distal end of the outer member may be welded to the distal end of the inner member. In some cases, the outer member may include an outer coiled member. In some cases, the outer member may include a plurality of distinct annular rings that are disposed about the inner member.

In some cases, the adjustment mechanism may include a removable adjustment mechanism. As an example, the adjustment mechanism may include reverse-action pliers. The reverse-action pliers may include first and second jaws that each include a U-shaped cutout to accommodate the inner member therethrough. The reverse-action pliers may further include first and second handles. Moving the first and second handles closer together causes the first and second jaws to move farther apart, for example. In some cases, the inner member may include a threaded section and the adjustment mechanism may include a threaded fastener that is threadedly engaged with the threaded section, such that rotation of the adjustment mechanism adjusts compression of the outer member. In some cases, the threaded fastener may be textured to improve grip.

In some instances, a guidewire includes an outer coiled member and an inner member that extends within the outer coiled member. A distal end of the outer coiled member is constrained from translating relative to a distal end of the inner member. An adjustment mechanism is adapted to urge a proximal end of the outer coiled member distally relative to the proximal end of the inner member in order to increase a stiffness of the guidewire. The adjustment mechanism is adapted to allow the proximal end of the outer coiled member to move proximally relative to the proximal end of the inner member in order to decrease the stiffness of the guidewire.

In some cases, the distal end of the outer coiled member may also be constrained from rotating relative to the distal end of the inner member, i.e., constrained from both translation and rotation. In some cases, the adjustment mechanism may include reverse-action pliers. The reverse-action pliers include first and second jaws that each have a U-shaped cutout to accommodate the inner member therethrough. The reverse-action pliers also include first and second handles. Moving the first and second handles closer together may cause the first and second jaws to move farther apart. In some cases, the inner member includes a threaded section and the adjustment mechanism may include a threaded fastener that is threadedly engaged with the threaded section such that rotation of the adjustment mechanism adjusts compression of the outer coiled member.

In some instances, a guidewire includes an outer coiled member and an inner member that extends within the outer coiled member. A distal end of the outer coiled member is welded to a distal end of the inner member. A removable adjustment mechanism is adapted to selectively apply a compressive force to a proximal end of the outer coiled member. The removable adjustment mechanism is adapted to be removed from the guidewire to accommodate device exchanges over the guidewire. The guidewire increases in stiffness when the removable adjustment mechanism applies a compressive force to the proximal end of the outer member.

In some cases, the removable adjustment mechanism includes a first pliers member having a first jaw with a U-shaped cutout and a first handle, and a second pliers member having a second jaw with a second U-shaped cutout and a second handle. The second pliers member may be pivotably secured relative to the first pliers member. Urging the first and second handles towards each other may cause the first and second jaws to move away from each other. In some cases, applying a compressive force to the proximal end of the outer member may also place a tensile force on the proximal end of the inner member.

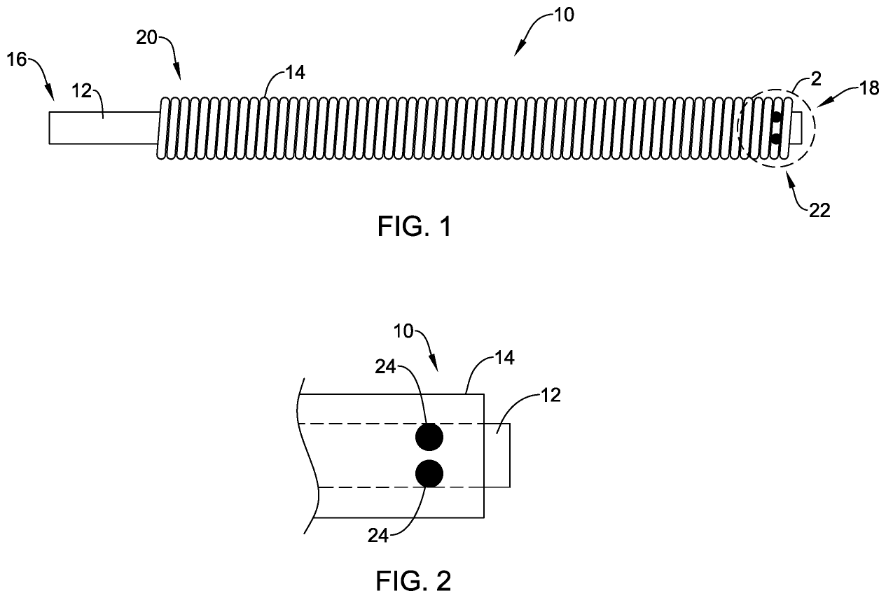

FIG. 1 is a schematic view of an illustrative guidewire 10. The illustrative guidewire 10 may be considered as being adapted or configured to be able to exhibit a varying stiffness or flexibility, depending on the requirements of the particular anatomy through which the guidewire 10 is being advanced, and depending on whether the guidewire is being advanced through the anatomy or other devices are being advanced over the guidewire 10. In some cases, there is a desire for the guidewire 10 to be flexible, particularly when navigating a complex or tortuous path, or when navigating soft tissue that could otherwise easily be perforated by the guidewire 10. In some cases, there is a desire for the guidewire 10 to be stiffer, such as for rail support during device exchange and pushability. The guidewire 10 includes an inner member 12 and an outer member 14. In some cases, the inner member 12 may be a core wire made of nitinol, for example. As an example, the inner member 12 may have a diameter of 0.010 inches to 0.025 inches. In some cases, the outer member 14 may be an outer coiled member such as a spring coil made of any suitable metal. As an example, the spring coil may be formed from a wire having a diameter of 0.003 inches to 0.006 inches, or as high as 0.010 inches. The inner member 12 extends from a proximal end 16 to a distal end 18. The outer member 14 extends from a proximal end 20 to a distal end 22. In some cases, the distal end 22 of the outer member 14 is constrained from translating relative to the distal end 18 of the inner member 12. In some cases, the distal end 22 of the outer member 14 may also be constrained from rotating relative to the distal end 18 of the inner member 12. However, the proximal end 20 of the outer member 14 is not constrained from moving, in either a translational direction or in a rotational direction, relative to the proximal end 16 of the inner member 12 in order to allow for compression and decompression of the outer member 14. In some cases, compression of the outer member 14 stiffens the guidewire 10 while decompression of the outer member 14 brings the guidewire 10 back into a relaxed state allowing for greater flexibility.

In FIG. 1, the distal end 22 of the outer member 14 is constrained from both translational and rotational movement relative to the distal end 18 of the inner member 12. As seen in FIG. 2, which is an enlarged schematic view of a portion of the guidewire 10, the distal end 22 of the outer member 14 is secured relative to the distal end 18 of the inner member 12 via one or more welds 24. While two welds 24 are visible, there may be additional welds 24 disposed circumferentially around the outer member 14. In some cases, the welds 24 may be distinct point welds, as shown. In some cases, the welds 24 may be elongated, and may extend circumferentially at least part way around the outer member 14, or may extend all of the way around the outer member 14.

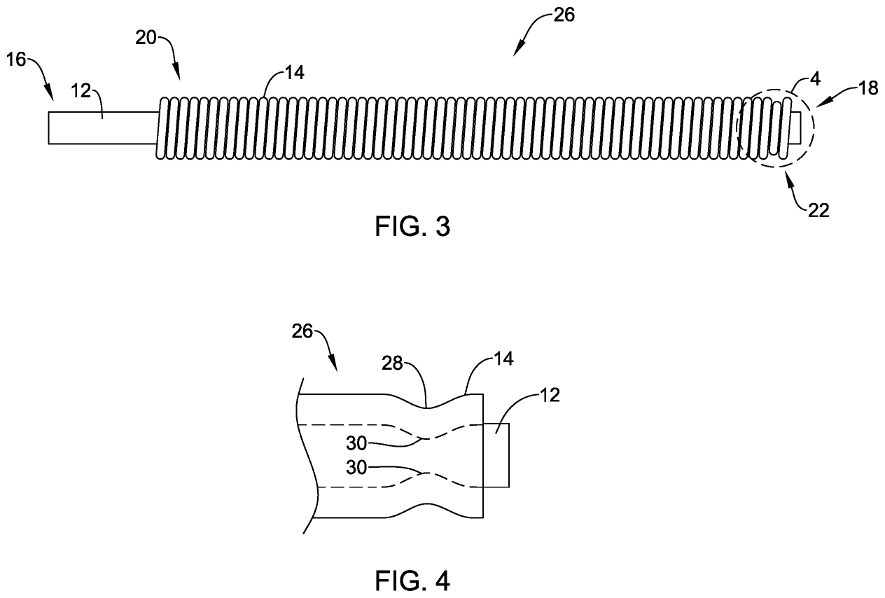

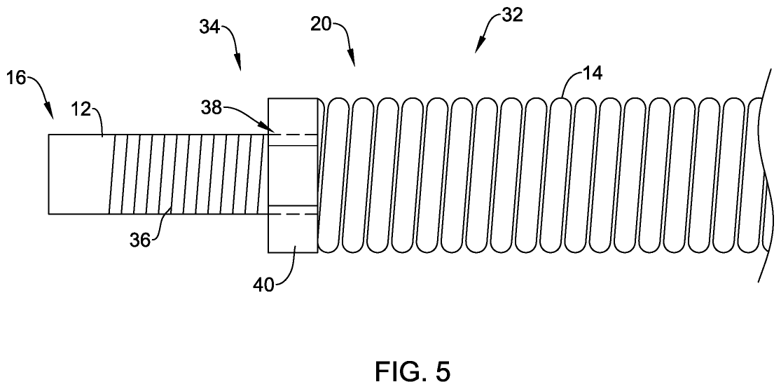

FIG. 3 is a schematic view of an illustrative guidewire 26 that is similar to the guidewire 10. However, in the illustrative guidewire 26, the distal end 22 of the outer member 14 is constrained from translational movement relative to the distal end 18 of the inner member 12 but is not constrained from relative rotational movement. In the guidewire 26, the distal end 22 of the outer member 14 may be allowed to rotate relative to the distal end 18 of the inner member 12. As seen in FIG. 4, which is an enlarged schematic view of a portion of the guidewire 26, the distal end 22 of the outer member 14 is secured relative to the distal end 18 of the inner member 12 via a crimp 28 that forces the outer member 14 into the inner member 12. In some cases, crimping the outer member 14 onto the inner member 12 results in a crimp 30 formed within the inner member 12. In some cases, the inner member 12 may be formed having the crimp 30, and the crimp 28 may subsequently be formed within the outer member 14. The crimp 28 prevents relative translational movement between the outer member 14 and the inner member 12 but may in some cases permit rotational movement between the outer member 14 and the inner member 12.

The guidewires 10 and 26 include an adjustment mechanism that may be used to control the relative position of the proximal end 20 of the outer member 14 relative to the proximal end 16 of the inner member 12. Compressing the outer member 14 by moving the proximal end 20 of the outer member 14 relative to the proximal end 16 of the inner member 12 can stiffen the guidewires 10 and 26, while allowing the proximal end 20 of the outer member 14 to move freely relative to the proximal end 16 of the inner member 12 can allow the guidewires 10 and 26 to become more flexible. In some cases, an adjustment mechanism may be part of the guidewires 10 and 26, and may remain as part of the guidewires 10 and 26 throughout use of the guidewires 10 and 26. In some cases, an adjustment mechanism may be a separate tool, and may be coupled to the guidewires 10 and 26 when there is a desire to stiffen the guidewires 10 and 26, and may be removed from the guidewires 10 and 26 when there is a desire to allow the guidewires 10 and 26 to be more flexible, or when there is a need to advance another device over the guidewires 10 and 26.

FIG. 5 is a schematic view of a proximal portion of an illustrative guidewire 32. The illustrative guidewire 32 may be considered as being an example of either the guidewire 10 or the guidewire 26. The guidewire 32 includes an adjustment mechanism 34. In some cases, as shown, the inner member 12 may include a threaded section 36. The adjustment mechanism 34 may include a threaded fastener 38 that is threadedly engaged with the threaded section 36. Rotating the threaded fastener 38 in a first direction may cause the threaded fastener 38 to move distally, thereby applying a compressive force to the outer member 14, thereby compressing the outer member 14. In some cases, the outer member 14 may only be partially compressed, meaning that there may still be space between adjacent coil windings of the outer member 14. In some cases, applying a compressive force to the outer member 14 also applies a tensile force to the inner member 12 as the threaded fastener 38 braces against the threaded section 36 of the inner member 12 as the threaded fastener 38 is rotated and applies a compressive force to the outer member 14. This is particularly true once the outer member 14 is fully compressed, e.g., adjacent coil windings of the outer member 14 are fully in contact. As the outer member 14 is compressed, the outer member 14 is less able to bend and flex, and thereby the guidewire 32 becomes stiffer (or less flexible). Rotating the threaded fastener 38 in a second, opposing, direction may cause the threaded fastener 38 to move proximally, thereby allowing the outer member 14 to relax from a compressed state. As the outer member 14 is allowed to relax, the outer member 14 is more able to bend and flex, and thereby the guidewire 32 becomes less stiff (or more flexible).

In some cases, an outer surface 40 of the threaded fastener 38 may be knurled or textured in order to make it easier for a user to grasp and rotate the threaded fastener 38. In some cases, the outer surface 40 of the threaded fastener 38 may also be elongated, in order to make it easier for a user to grasp the threaded fastener 38. Alternatively, the outer surface 40 of the threaded fastener 38 may be fashioned to emulate the shape of a four-sided or a six-sided nut such that the threaded fastener 38 may easily be engaged and rotated using a tool similar to a wrench or a socket. As an example, an open-ended wrench may be used to engage and rotate the threaded fastener 38, and can easily be removed when there is no desire to stiffen the guidewire 32, or when there is a desire to exchange devices over the guidewire 32.

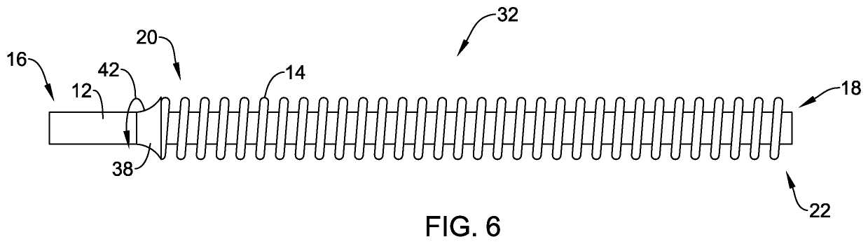

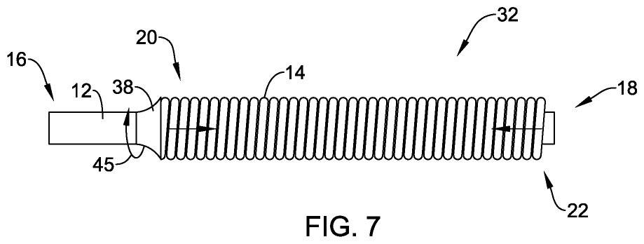

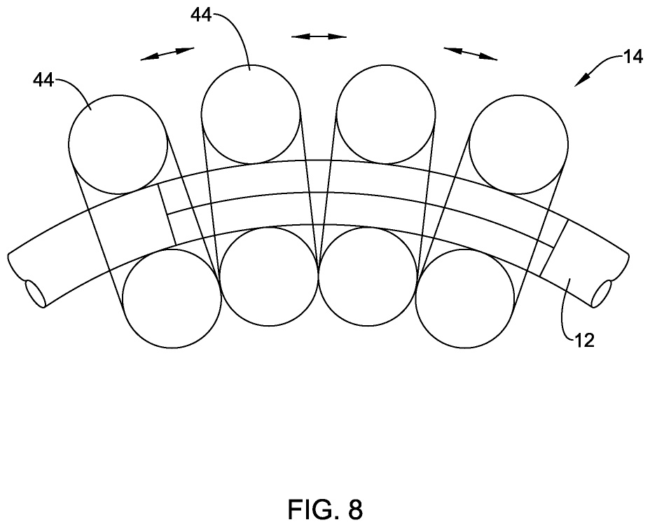

FIG. 6 shows the guidewire 32 with its outer member 14 in a relaxed configuration (closed coil in its relaxed state) while FIG. 7 shows the guidewire 32 with its outer member 14 in a compressed configuration. It will be appreciated that the guidewire 32 may be placed into a plurality of different configurations that are intermediate the two configurations shown in FIGS. 6 and 8. The guidewire 32 will be relatively more flexible in the configuration shown in FIG. 6 and will be relatively stiffer in the configuration shown in FIG. 7. In FIG. 6, the threaded fastener 38 has been rotated in a direction indicated by an arrow 42, which has caused the threaded fastener 38 to move proximally relative to the inner member 12 and which has allowed the outer member 14 to relax. In FIG. 7, the threaded fastener 38 has been rotated in a direction indicated by an arrow 44, which has caused the threaded fastener 38 to move distally relative to the inner member 12, thereby compressing the outer member 14 (and placing the inner member 12 in tension). It will be appreciated that the relative rotational direction necessary to cause the threaded fastener 38 to move proximally or distally may depend on whether the threaded section 36 and an interior threaded surface (not shown) of the threaded fastener 38 have a right-handed thread or a left-handed thread.

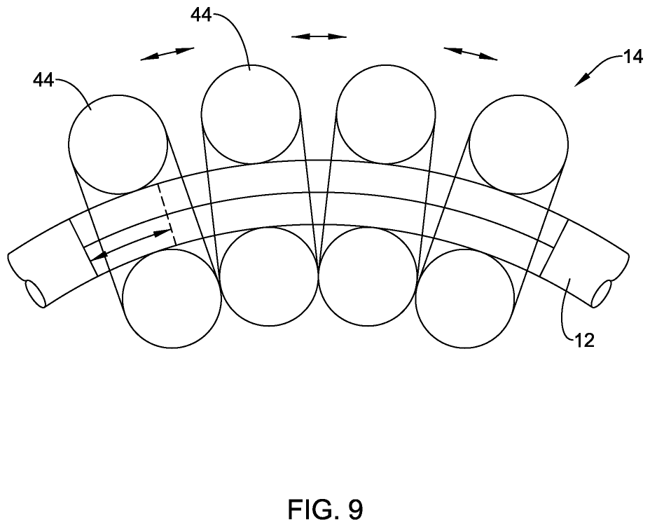

FIG. 8 provides an illustration of how the guidewire 32 behaves when the outer member 14 is not translationally (or rotationally) constrained against movement relative to the inner member 12, and FIG. 9 provides an illustration of how the guidewire 32 behaves when the outer member 14 is translationally (and optionally rotationally) constrained against movement relative to the inner member 12. As shown in FIG. 8, when the guidewire 32 enters a bending state and the outer member 14 is not constrained at its proximal end, the coil (outer member 14) on the outside of the bending radius opens and the coil (outer member 14) slides over the inner member 12 as the coil (outer member 14) elongates. The inner member 12 may also bend to a similar bending radius. When the coil (outer member 14) is not constrained, the bending force may be described by the equation:

Total Bending Force=Force¿Bend Inner+Force¿Bend/Spread Coil.

As shown in FIG. 9, when the guidewire 32 enters a bending state and the outer member 14 is constrained from moving relative to the inner member 12, the coil on the outside bending radius opens as before. However, the outer member 14 is not allowed to slide freely over the inner member 12 as the outer member 14 elongates. In this case, the inner member 12 is forced to elongate or stretch as the inner member 14 also bends to a similar bending radius. Accordingly, the overall bending force increases, and may be described by the equation:

Total Bending Force=Force¿Bend Inner+Force¿Bend/Spread Coil+Force¿Elongate Inner Member.

The additional force needed to elastically elongate the inner member 12 to accommodate the expansion of the coil (outer member 14) results in a stiffer guidewire. In some cases, having the coil (outer member 14) partially expanded may help in fine tuning the necessary elongation or stretch of the inner member 12.

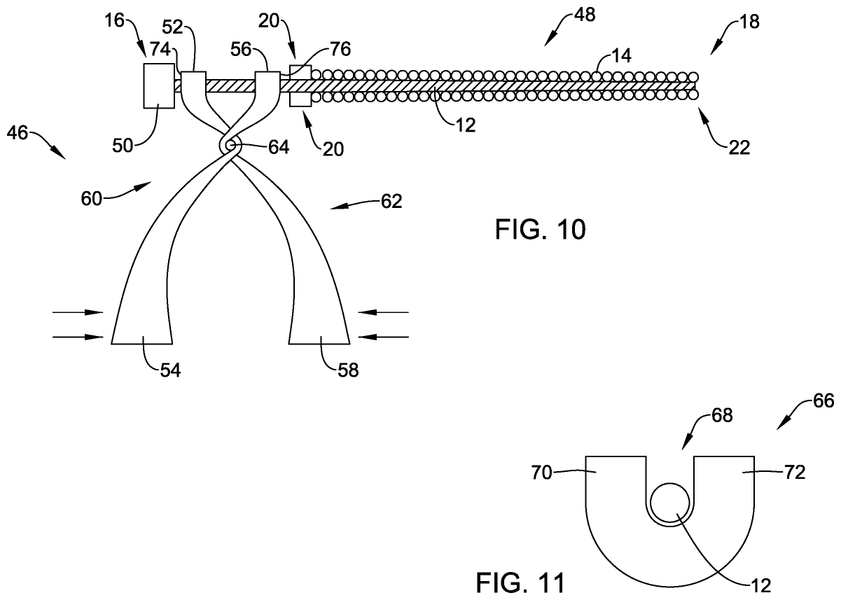

In some cases, the adjustment mechanism may be a removable adjustment mechanism. FIG. 10 shows an illustrative removable adjustment mechanism 46 shown in combination with an illustrative guidewire 48. The illustrative guidewire 48 may be considered as representing any of the guidewires 10, 26, and 32, and includes the outer member 14 extending over the inner member 12. In some cases, the inner member 12 may include a widened section 50 that allows the removable adjustment mechanism 46 to engage the proximal end 16 of the inner member 12. The removable adjustment mechanism 46 includes a first jaw 52 that is coupled with a first handle 54 and a second jaw 56 that is coupled with a second handle 58. In some cases, the first jaw 52 and the first handle 54 may in combination be referred to as a first pliers member 60 and the second jaw 56 and the second handle 58 may in combination be referred to as a second pliers member 60. In some cases, the first pliers member 60 is a unitary structure including both the first jaw 52 and the first handle 54. In some cases, the second pliers member 62 is a unitary structure including both the second jaw 56 and the second handle 58. In some cases, the first pliers member 60 and the second pliers member 62 are pivotably coupled together at a pivot 64. In some cases, the removable adjustment mechanism 46 may be considered as being a reverse-action pliers, meaning that when the first handle 54 and the second handle 58 are moved closer together, the first jaw 52 and the second jaw 56 move farther apart. Conversely, moving the first handle 54 and the second handle 58 farther apart causes the first jaw 52 and the second jaw 56 to move closer together.

FIG. 11 is a schematic cross-sectional view through a jaw 66 that may be considered as representing either of the first jaw 52 or the second jaw 56. As can be seen, the jaw 66 includes a U-shaped cutout 68, giving the jaw 66 a first segment 70 on a first side of the inner member 12 and a second segment 72 on a second side of the inner member 12. Referring back to FIG. 10, by engaging the removable adjustment mechanism 46 with the inner member 12, an outer surface 74 of the first jaw 52 engages the widened section 50 of the inner member 12 and is able to push against the widened section 50 of the inner member 12, and an outer surface 76 of the second jaw 56 is able to push against the proximal end 20 of the outer member 14. Squeezing the first handle 54 and the second handle 58 closer together causes the first jaw 52 and the second jaw 56 to move farther apart. As the first jaw 52 and the second jaw 56 move farther apart, a compressive force is applied to the outer member 14 (and a concurrent tensile force is applied to the inner member 12), thereby stiffening the guidewire 48.

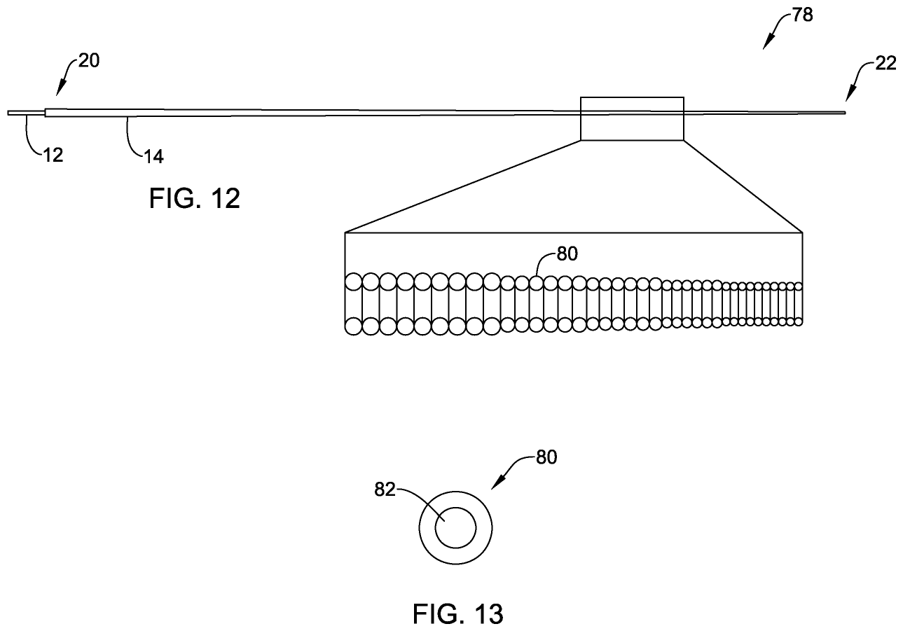

FIG. 12 is a schematic view of an illustrative guidewire 78 that may be considered as being an example of any of the guidewires 10, 26, and 32. The guidewire 78 includes the inner member 12 and the outer member 14. In this case, however, the outer member 14 is not a spring coil, but rather includes a plurality of distinct annular rings 80 that are arranged side by side each other along the length of the inner member 12. In some cases, the annular rings 80 may vary in diameter along a length of the outer member 14, with the annular rings 80 having a greater diameter closer to the proximal end 20 of the outer member 14 and the annular rings 80 having a reduced diameter closer to the distal end 22 of the outer member 14. In some cases, the annular rings 80 may have a plurality of diameter changes along the length of the outer member 14. As seen in FIG. 13, each of the distinct annular rings 80 define an aperture 82 extending therethrough that is sized to slidingly accommodate the inner member 12 therethrough. With the distinct annular rings 80 arranged side by side, defining a length of the outer member 14, all of the apertures 82 will align together, forming a lumen that accommodates the inner member 12.

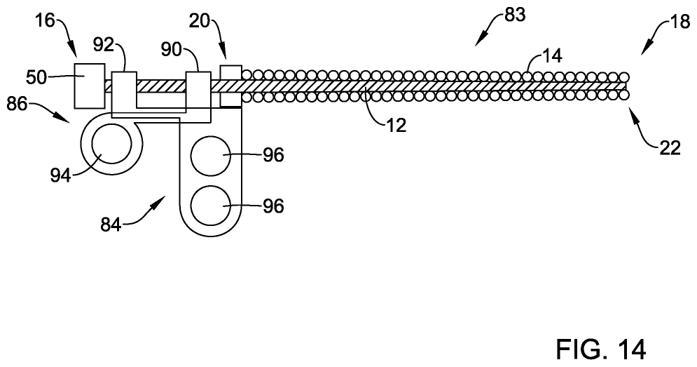

FIG. 14 shows an illustrative removable adjustment mechanism 84 shown in combination with an illustrative guidewire 83. The illustrative guidewire 83 may be considered as representing any of the guidewires 10, 26, and 32, and includes the outer member 14 extending over the inner member 12. In some cases, the inner member 12 may include a widened section 50 that allows the removable adjustment mechanism 84 to engage the proximal end 16 of the inner member 12. The removable adjustment mechanism 84 includes a first member 86 and a second member 88. In some cases, the first member 86 includes a portion 90 that is configured to push on the proximal end 20 of the outer member 14. In some cases, the second member 88 includes a portion 92 that is configured to engage the widened portion 50 of the inner member 12. Together, the first member 86 and the second member 88 are configured to be easily grasped and manipulated by holding the removable adjustment mechanism 84 in one hand. The first member 86 and the second member 88 may together be considered as forming a finger loop tensioner. The first member 86 includes an opening 94 that may accommodate a thumb, for example, and the second member 88 includes openings 96 that may accommodate two fingers. Moving the first member 86 and the second member 88 closer together causes a compressive force to be applied to the outer member 14 and a tensile force to be applied to the inner member 12.

The guidewires 10, 26, 32, 48, 78, and 83 may be used in a variety of medical procedures. As an example, any of the guidewires 10, 26, 32, 48, 78, and 83 may be used when performing procedures within the biliary tract. The guidewire 10, 26, 32, 48, 78, and 83 may be advanced into the anatomy. As needed, the relative stiffness of the guidewire 10, 26, 32, 48, 78, and 83 may be navigated through a tortuous anatomy using a lower stiffness configuration in which the proximal end 20 of the outer member 14 is able to move relative to the proximal end 16 of the inner member 12. In some cases, there may be a desire to increase the stiffness of the 10, 26, 32, 48, 78, and 83 in order to provide rail support for advancing other devices over the guidewire 10, 26, 32, 48, 78, and 83. The stiffness of the guidewire 10, 26, 32, 48, 78, and 83 may be increased by applying tension to the inner member 12 and applying compression 14 to the outer member 14, thereby moving the proximal end 20 of the outer member 14 in a distal direction. In some cases, there may be a desire to decrease the stiffness of the guidewire 10, 26, 32, 48, 78, and 83 prior to advancing through a particularly sensitive portion of the anatomy in order to avoid possible perforations. The stiffness of the guidewire 10, 26, 32, 48, 78, and 83 may be reduced by reducing the tension applied to the inner member 12 and the compression applied to the outer member 14. This may be achieved by using an adjustment mechanism such as the adjustment mechanism 38 or the removable adjustment mechanism 46.

The materials that can be used for the various components of the systems presently disclosed may include those commonly associated with medical devices. The devices and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS®400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility may be imparted. For example, the devices described herein, or portions thereof, may be made of a material that does not substantially distort the image and create substantial artifacts (i.e., gaps in the image). Certain ferromagnetic materials, for example, may not be suitable because they may create artifacts in an MRI image. The devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A guidewire, comprising:
an outer member;
an inner member extending within the outer member;
wherein a distal end of the outer member is constrained from translating relative to a distal end of the inner member; and
an adjustment mechanism adapted to selectively apply a compressive force to a proximal end of the outer member;
wherein the guidewire increases in stiffness when the adjustment mechanism applies a compressive force to the proximal end of the outer member.

2. The guidewire of claim 1, wherein applying a compressive force to the proximal end of the outer member also places a tensile force on the proximal end of the inner member.

3. The guidewire of claim 1, wherein the distal end of the outer member is crimped onto the distal end of the inner member.

4. The guidewire of claim 1, wherein the distal end of the outer member is also constrained from rotating relative to the distal end of the inner member.

5. The guidewire of claim 1, wherein the distal end of the outer member is welded to the distal end of the inner member.

6. The guidewire of claim 1, wherein the outer member comprises an outer coiled member.

7. The guidewire of claim 1, wherein the outer member comprises a plurality of distinct annular rings disposed about the inner member.

8. The guidewire of claim 1, wherein the adjustment mechanism comprises a removable adjustment mechanism.

9. The guidewire of claim 1, wherein the adjustment mechanism comprises reverse-action pliers.

10. The guidewire of claim 9, wherein the reverse-action pliers comprise first and second jaws each including a U-shaped cutout to accommodate the inner member therethrough.

11. The guidewire of claim 10, wherein the reverse-action pliers further comprise first and second handles, where moving the first and second handles closer together causes the first and second jaws to move farther apart.

12. The guidewire of claim 1, wherein the inner member includes a threaded section and the adjustment mechanism comprises a threaded fastener that is threadedly engaged with the threaded section, such that rotation of the adjustment mechanism adjusts compression of the outer member.

13. The guidewire of claim 12, wherein the threaded fastener is textured to improve grip.

14. A guidewire, comprising:
an outer coiled member;
an inner member extending within the outer coiled member;
wherein a distal end of the outer coiled member is constrained from translating relative to a distal end of the inner member; and
an adjustment mechanism adapted to:
urge a proximal end of the outer coiled member distally relative to the proximal end of the inner member in order to increase a stiffness of the guidewire; and to
allow the proximal end of the outer coiled member to move proximally relative to the proximal end of the inner member in order to decrease the stiffness of the guidewire.

15. The guidewire of claim 14, wherein the distal end of the outer coiled member is constrained from rotating relative to the distal end of the inner member.

16. The guidewire of claim 14, wherein the adjustment mechanism comprises reverse-action pliers including:
first and second jaws each including a U-shaped cutout to accommodate the inner member therethrough; and
first and second handles, where moving the first and second handles closer together causes the first and second jaws to move farther apart.

17. The guidewire of claim 14, wherein the inner member includes a threaded section and the adjustment mechanism comprises a threaded fastener that is threadedly engaged with the threaded section, such that rotation of the adjustment mechanism adjusts compression of the outer coiled member.

18. A guidewire, comprising:
an outer coiled member;
an inner member extending within the outer coiled member;
wherein a distal end of the outer coiled member is welded to a distal end of the inner member; and
a removable adjustment mechanism adapted to:
selectively apply a compressive force to a proximal end of the outer coiled member; and
be removed from the guidewire to accommodate device exchanges over the guidewire;
wherein the guidewire increases in stiffness when the removable adjustment mechanism applies a compressive force to the proximal end of the outer member.

19. The guidewire of claim 18, wherein the removable adjustment mechanism comprises:
a first pliers member including a first jaw with a U-shaped cutout and a first handle; and
a second pliers member including a second jaw with a second U-shaped cutout and a second handle, the second pliers member pivotably secured relative to the first pliers member;
wherein urging the first and second handles towards each other causes the first and second jaws to move away from each other.

20. The guidewire of claim 18, wherein the removable adjustment mechanism comprises:
a first member configured to engage a proximal end of the inner member, the first member including one or more finger loops; and
a second member configured to engage a proximal end of the outer member, the second member including one or more finger loops;
wherein moving the first member and the second member closer together applies a compressive force to the outer member and a tensile force to the inner member.
